(19) United States
(12) Patent Application Publication
(10) Pub. No.: US 2025/0375090 A1
(43) Pub. Date: Dec. 11, 2025

(54) HIGHLY INTEGRATED DETACHABLE ELECTRONIC ENDOSCOPE

(71) Applicant: ZOLWISE (WUHAN) TECHNOLOGY CO., LTD, Wuhan (CN)

(72) Inventors: Jungang MAO, Wuhan (CN); Liang GU, Wuhan (CN); Suyun LI, Wuhan (CN); Liqiong LI, Wuhan (CN)

(73) Assignee: ZOLWISE (WUHAN) TECHNOLOGY CO., LTD, Wuhan (CN)

(21) Appl. No.: 19/300,538

(22) Filed: Aug. 14, 2025

Related U.S. Application Data
(63) Continuation of application No. PCT/CN2024/077425, filed on Feb. 18, 2024.

(30) Foreign Application Priority Data
May 15, 2023 (CN) 202310542378.3
May 31, 2023 (CN) 202310632565.0
May 31, 2023 (CN) 202321360009.4
Jul. 21, 2023 (CN) 202310899396.7
Jul. 21, 2023 (CN) 202310899401.4

(51) Int. Cl. A61B 1/00; A61B 1/045; A61B 1/05; A61B 1/06; A61B 90/00

(52) U.S. Cl. CPC A61B 1/00006; A61B 1/00016; A61B 1/0002; A61B 1/0004; A61B 1/00048; A61B 1/00066; A61B 1/00114; A61B 1/00124; A61B 1/00128; A61B 1/045; A61B 1/05; A61B 1/0676; A61B 90/03; A61B 90/08; A61B 2090/034; A61B 2090/0803

(57) ABSTRACT

A highly integrated detachable electronic endoscope includes an endoscope body and a handle that are detachably connected via a connector. The endoscope body is configured for insertion into an examined body, and a camera module and an illumination module are disposed within a front end of the endoscope body. An image processing module is disposed inside the handle, while an operation key is disposed outside the handle, and the handle is connected to an external display via a cable. A limiter or an electronic control component allowing customization of a number of uses is disposed on the endoscope body, the handle, or the connector. The highly integrated detachable electronic endoscope features high integration level, high safety, good continuous usability, and low cost.

### CROSS-REFERENCE TO RELATED APPLICATIONS

This application is a continuation of International Patent Application No. PCT/CN2024/077425 with a filing date of Feb. 18, 2024, designating the United States, now pending, and further claims priorities to Chinese Patent Application No. 202320227678.8 with a filing date of Feb. 15, 2023, Chinese Patent Application No. 202310121259.0 with a filing date of Feb. 15, 2023,Chinese Patent Application No. 202310542378.3 with a filing date of May 15, 2023,Chinese Patent Application No. 202321360009.4 with a filing date of May 31, 2023,Chinese Patent Application No. 202310632565.0 with a filing date of May 31, 2023,Chinese Patent Application No. 202310899396.7 with a filing date of Jul. 21, 2023, and Chinese Patent Application No. 202310899401.4 with a filing date of Jul. 21, 2023. The content of the aforementioned applications, including any intervening amendments thereto, is incorporated herein by reference.

### TECHNICAL FIELD

The present disclosure relates to the field of medical devices, and in particular, to a highly integrated detachable electronic endoscope.

### BACKGROUND

With the increasing global adoption of minimally invasive surgery, the endoscope industry has entered a phase of rapid development. Continuous advancements in minimally invasive surgery have driven the diversification of endoscope types, including electronic endoscopes.

Traditional electronic endoscopes, similar to optical endoscopes, require supporting equipment such as carts, light sources, image processing hosts, and monitors, resulting in a complex and expensive system which, in practical use, presents great difficulties in transportation, installation, use, and maintenance.

Moreover, sterilization remains a critical issue in the field of endoscopes, encompassing the following key challenges: The first one is sterilization efficacy. Electronic endoscopes include therein electronic components that are sensitive to high temperatures. For this reason, hydrogen peroxide low-temperature plasma sterilization is generally adopted in the industry at present. However, compared to traditional high-temperature steam sterilization, this method relies on a complex mechanism involving oxidizing agents, ultraviolet (UV) light, and high-energy particles to achieve desired sterilization effects. Issues such as equipment aging, oxidizing agent degradation, or insufficient plasma generation in hospitals can compromise sterilization efficacy, leading to incomplete sterilization which is a major cause of cross-infection in patients. The second one is sterilization resistance. The hydrogen peroxide used in hydrogen peroxide low-temperature plasma sterilization is a strong oxidizing agent and generates UV radiation that accelerates material aging. If standard engineering plastics or rubber are used, aging failure may occur after 50 to 100 sterilization cycles, resulting in equipment failure and a short service life. If key components are fabricated from special plastics, such as polyether ether ketone (PEEK) and polyphenylsulfone (PPSU), or fluororubber, while their service life will be prolonged, their production costs can significantly increase. The third one is sterilization costs, which include time and consumable expenses. The low-temperature plasma sterilization used for electronic endoscopes requires thorough cleaning and drying prior to sterilization. A typical reprocessing cycle takes 1.5 to 2 hours, with the primary consumable being hydrogen peroxide sterilizing agent. The consumable cost per low-temperature plasma sterilization cycle ranges approximately RMB 30 to 100, varying depending on the sterilizer type, the operational mode, and the inner chamber space. These three sterilization challenges raise the barriers to adoption of electronic endoscopes in hospitals.

Even without sterilization concerns, the service life of electronic endoscopes remains problematic. Given the high cost of equipment, hospitals typically expect electronic endoscopes to be used for 5 to 8 years or longer. During this process, lens wear, endoscope body deformation, and thermal aging of internal components can lead to performance degradation of the equipment and compromise effects in use.

In comprehensive consideration of the aforementioned challenges in the field of electronic endoscopes, there exists a significant market demand for a highly integrated electronic endoscope that eliminates sterilization requirements and prevents overheating, electrical leakage, and connection failures. With such an electronic endoscope, the barriers to adoption of electronic endoscopes can be greatly lowered, and it is conducive to further popularize the endoscopic surgery in underdeveloped areas.

### SUMMARY OF PRESENT INVENTION

A major objective of the present disclosure is to provide a highly integrated detachable electronic endoscope with a limitable number of uses.

The present disclosure provides the following technical solution.

A highly integrated detachable electronic endoscope is provided, including: an endoscope body and a handle that are detachably connected via a connector;
where the endoscope body is configured for insertion into an examined body, and a camera module and an illumination module are disposed within a front end of the endoscope body;
an image processing module is disposed inside the handle, while an operation key is disposed outside the handle, and the handle is connected to an external display via a cable; and
a limiter or an electronic control component allowing customization of a number of uses is disposed on the endoscope body, the handle, or the connector.

In the above technical solution, the endoscope body includes an outer endoscope tube and an inner endoscope rod independent of each other and capable of being assembled together; the outer endoscope tube has a front end on which protective glass is hermetically mounted, and an open rear end; the inner endoscope rod includes a front end serving as a mounting bracket, a hollow rod in a middle portion, and a rear end serving as the connector for connection with the handle; the camera module and the illumination module are mounted at a front end of the mounting bracket and limited by the protective glass; a rear end of the mounting bracket is connected to a front end of the hollow rod; the connector has a front end connected to a rear end of the hollow rod, a middle portion hermetically mounted at the rear end of the outer endoscope tube, and a rear end serving as a plug-in port and located on an outer side of the outer endoscope tube; a cable of the camera module and the illumination module passes through the hollow rod and is electrically connected to the connector; when the plug-in port is inserted, the camera module and the illumination module are connected to power and thus are enabled for use; the plug-in port is connected to the electronic control component; a number of uses is incremented by one every time when the plug-in port is inserted; and after a set number of uses is reached, when the plug-in port is inserted, the electronic control component controls the camera module and the illumination module to be disabled.

In the above technical solution, the front end of the connector is threadedly connected to the rear end of the hollow rod; or the hollow rod is made up of two threadedly connected rod sections to enable length adjustment of the inner endoscope rod.

In the above technical solution, the mounting bracket is an electrically insulating and thermally conductive bracket, and the hollow rod is a heat conduction tube.

In the above technical solution, a circuit is disposed within each of the endoscope body and the handle; the electronic control component includes a non-volatile memory chip disposed on the circuit within the endoscope body and a processing chip disposed on the circuit within the handle; the non-volatile memory chip is configured to store and encrypt information of a product manufacturer, a product serial number, and a number of uses; and the processing chip is configured to: after being installed in the handle and powered on, read and decrypt the information stored on the non-volatile memory chip; when the information meets requirements, activate the camera module, and update the information of the number of uses, and then encrypt and write back the information of the number of uses to the non-volatile memory chip; and when encryption is failed, the information does not meet the requirements, or a number of allowable uses is zero, not activate the camera module, or power off the circuit within the endoscope body.

In the above technical solution, a circuit is disposed within each of the endoscope body and the handle; the electronic control component includes a microcontroller unit (MCU) or single-chip microcomputer disposed on the circuit within the endoscope body and a processing chip disposed on the circuit within the handle; the MCU or single-chip microcomputer is configured to communicate with the processing chip via a customized communication protocol and to store information of a product manufacturer, a product serial number, and a number of uses; and the processing chip is configured to, after being installed in the endoscope body and powered on, read the information stored on the MCU or single-chip microcomputer; when the information meets requirements, activate the camera module, and modify the information of the number of uses via a protocol; and when the information does not meet the requirements or a number of allowable uses is zero, not activate the camera module, or power off the endoscope body.

In the above technical solution, a circuit is disposed within the endoscope body; the electronic control component includes a programmable logic device disposed on the circuit within the endoscope body; the programmable logic device includes a circuit of a state machine having preset transition states; the state machine is configured to undergo a transition in state every time when powered on; the transition states are unidirectional; and the state machine is further configured to permanently disconnect the circuit within the endoscope body when a transition to a last state occurs so that the endoscope body is incapable of being used anymore.

In the above technical solution, a circuit is disposed within each of the endoscope body and the handle; the electronic control component includes a memory or a microcontroller unit (MCU) or single-chip microcomputer disposed on the circuit within the endoscope body, and a processing chip and a wireless communication module disposed on the circuit within the handle; the memory or the MCU or single-chip microcomputer is configured to store information of a product serial number; the processing chip is configured to, after being installed in the endoscope body and powered on, read the information of the product serial number stored on the memory or the MCU or single-chip microcomputer, and upload the information of the product serial number to a cloud server via the wireless communication module; the cloud server is configured to register the information of the product serial number, and determine whether to distribute a product license according to previous usage logs of the endoscope body; when the wireless communication module receives a license to the present electronic endoscope, the wireless communication module transmits the license to the processing chip such that the processing chip activates the camera module; and when the wireless communication module receives a disable command, the wireless communication module transmits the disable command to the processing chip such that the processing chip does not activate the camera module, or powers off the endoscope body.

In the above technical solution, the limiter allowing customization of a number of uses is arranged on the connector, and includes a customization tool of a mechanical structure and an acting member; the customization tool includes a movable component capable of naturally expanding and a limiting component capable of retracting and limiting the movable component; when naturally expanding, the movable component is capable of hindering assembling of the connector; when the in movable component is retracted and limited by the limiting component, the connector is capable of being assembled smoothly; before assembling each time, the movable component is retracted and limited by the limiting component; and during assembling, the acting member acts upon the limiting component such that the limiting component releases the movable component.

In the above technical solution, the limiting component is disposable and replaceable; and during assembling, the limiting component is directly damaged by the acting member and thus is released.

In the above technical solution, the limiting component has an elastic piece limiting structure, the movable component is a spring leaf, and the acting member is an outer end face of an assembled object assembled with the connector; when the assembled object is not assembled with the connector, the spring leaf is blocked by the elastic piece limiting structure; and during assembling, the outer end face of the assembled object props against and removes the elastic piece limiting structure.

In the above technical solution, the limiting component is reusable and resettable; and during assembling, a state of the limiting component is changed by the acting member, enabling releasing by the limiting component.

In the above technical solution, the limiting component is a hooking slot, the movable component is composed of a spring leaf and a hook connected to the spring leaf, and the acting member is an acting surface of an assembled object assembled with the connector; when the assembled object is not assembled with the connector, the hook is hooked in the hooking slot such that the spring leaf is retracted and limited; and during assembling, the hook is pushed out of the hooking slot by the acting surface of the assembled object.

In the above technical solution, the connector includes a locking mechanism for locking the endoscope body, and the locking mechanism includes:
a locking rack provided with a through-hole cavity, where the through-hole cavity has a cross-sectional size matching an external size of the endoscope body; and in a cross-sectional direction, a plurality of fitting surfaces are present between the endoscope body and the through-hole cavity of the locking rack;
a locking driving component sleeved outside the locking rack in a rotary connection manner, where an inner wall of the locking driving component is provided with a locking surface and an unlocking surface in a rotation direction; and a minimum spacing between the locking surface and the endoscope body is smaller than a minimum spacing between the unlocking surface and the endoscope body; and

a locking component, where a limiting slot radially penetrating through the locking rack is formed in the locking rack, and the locking component is placed in the limiting slot and is capable of moving radially within the limiting slot; a size of the locking component ranges between the minimum spacing between the locking surface and the endoscope body and the minimum spacing between the unlocking surface and the endoscope body; and in a process of the locking driving component rotating on the locking rack, a surface of the locking driving component corresponding to the limiting slot is switched between the locking surface and the unlocking surface, thereby achieving locking and unlocking of the endoscope body.

In the above technical solution, at least two groups of locking components and limiting slots matching the locking components are arranged in a length direction of the endoscope body.

In the above technical solution, the locking component is a locking steel ball, and the limiting slot is cylindrical.

In the above technical solution, a transitional surface is provided between the locking surface and the unlocking surface; and the surface of the locking driving component corresponding to the limiting slot is switched among the unlocking surface, the transitional surface, and the locking surface.

In the above technical solution, a plurality of ball slots are formed in an outer wall of the locking rack; balls matching the ball slots are disposed within the ball slots; and the locking driving component is provided with a sliding surface for the balls to roll.

In the above technical solution, the highly integrated detachable electronic endoscope further includes a limiting step and a limiting groove matching each other, where the limiting groove is located in the outer wall of the locking rack, while the limiting step is fixedly arranged on the inner wall of the locking driving component, and the limiting step and the limiting groove are arranged axially; and the limiting groove and the limiting step are respectively located on a same side of the locking rack and the locking driving component.

In the above technical solution, two groups of force arm structures are disposed on the locking driving component and are distributed on left and right sides of an outer wall of the locking driving component; and the locking driving component is capable of increasing arm of force of the two groups of force arm structures such that an external force required by a locking operation is reduced.

The present disclosure has the following beneficial effects: without external cold light source and image processing host, the highly integrated detachable electronic endoscope of the present disclosure features a small size and a low weight, can be transported, transfer, assembled, and disassembled conveniently and efficiently, and is applicable to a plurality of usage scenarios such as hospitals, battlefields, and outdoors. This endoscope is designed to be customizable with the number of uses. Not only can this endoscope be made into the form of a disposable and sterile product, eliminating the risk of cross-infection caused by incomplete sterilization in hospitals, but also the number of its uses can be strictly limited, avoiding continuous use of the aged endoscope in some hospitals and further eliminating the risks relating to bio-safety and sterilization failure.

Further, a detachable design is adopted for the endoscope body such that the endoscope body is formed by assembling the independent outer endoscope tube and inner endoscope rod. The inner endoscope rod is formed by assembling the camera module, the illumination module, the mounting bracket, the hollow rod, the cable, and the connector. All such modules can be independent. The inner endoscope rod is adjustable in length and simple to assemble, exhibits high expansibility and portability, and can be applied to different specifications and types of endoscopes. The endoscope body can be customized with a set number of uses. After the set number of uses is reached, the inner endoscope rod can be taken out, and the camera module and the illumination module on the mounting bracket can be recycled. Furthermore, whether the mounting bracket, the hollow rod, and the connector are recycled can be selected according to an actual situation, thereby further reducing the cost.

Further, with the electrically insulating and thermally conductive bracket, good heat dissipation and electrical insulation of the product can be achieved, and overheating, electrical leakage, or failure under a high voltage can be avoided, thereby further guaranteeing patient's safety.

Further, the detachable endoscope body can be replaced rapidly. The whole electronic endoscope can be used in surgeries again as long as the handle is simply sterilized. Thus, the working efficiency of the equipment is greatly improved. During a same surgery, different models of endoscope bodies can be rapidly replaced to meet surgical requirements. moreover, the detachable endoscope body can prolong the overall service life of the equipment, thereby reducing the cost of the electronic endoscope, lower the barriers to use, and facilitating further promotion of the minimally invasive surgery.

Further, the electronic control component can track the usage state of the endoscope body. After the set number of uses is reached, further use is prohibited forcibly, thereby avoiding cross-contamination possibly caused by use beyond the limitation. The number of uses is controlled by the electronic control component and can be customized according to particular customers. For financially eligible patients, the endoscope body in contact with the human body is customized as one-time use and will be abandoned after use, and a new endoscope body may be used for next operation. For financially disadvantaged patients, the endoscope body is customized as multiple uses, and after each use, the endoscope body is sterilized. The number of uses may be changed according to situations, but is controlled within a ranged allowed by the sterilization efficacy.

Further, the locking component is disposed on the locking rack. By rotating the locking driving component, the surface of the locking driving component corresponding to the limiting slot can be switched between the locking surface and the unlocking surface. Since the minimum spacing between the locking surface and the locked part is smaller than the minimum spacing between the unlocking surface and the locked part, when the surface of the locking driving component corresponding to the limiting slot is switched from the unlocking surface to the locking surface, the locking component is gradually clamped between the locking surface and the locked part. At this point, the locking component exerts a certain pressure on the outer surface of the locked part, and the locking component works in cooperation with the locking rack, together with the plurality of fitting surfaces of the locked part, to lock the endoscope body. Conversely, when the surface of the locking driving component corresponding to the limiting slot is switched from the locking surface to the unlocking surface, the locking component cannot come into contact with the unlocking surface and the locked part at the same time, and exerts no pressure on the locked part, and the locked part can move within the locking rack. Based on the aforementioned measured, the endoscope body can be unlocked and locked rapidly. Moreover, the locking force is provided by the structural component, and is large, allowing for stable locking performance. In addition, this endoscope has the advantages of simple structure, integrated functionality of components, a reduced total number of components, and high reliability.

Certainly, the implementation of any product in the present disclosure does not necessarily need to achieve all of the above advantages.

### BRIEF DESCRIPTION OF THE DRAWINGS

To describe the technical solutions in the embodiments of the present disclosure or in the prior art more clearly, the following briefly describes the drawings required for describing the embodiments or the prior art. Apparently, the drawings in the following description show some embodiments of the present disclosure, and a person of ordinary skill in the art may still derive other drawings from these drawings without creative efforts.

FIG. 1 is a schematic diagram showing an application of a highly integrated detachable electronic endoscope according to an embodiment of the present disclosure;

FIG. 2 is a schematic structural diagram of a highly integrated detachable electronic endoscope according to an embodiment of the present disclosure;

FIG. 3 is a schematic structural diagram of an endoscope body of an electronic endoscope according to an embodiment of the present disclosure;

FIG. 4 a cross-sectional schematic diagram of an endoscope body of an electronic endoscope according to an embodiment of the present disclosure;

FIG. 5A is a schematic structural diagram I of a mounting bracket according to an embodiment of the present disclosure;

FIG. 5B is a schematic structural diagram II of the mounting bracket according to the embodiment of the present disclosure;

FIG. 6 is a schematic structural diagram of a heat conduction tube of an electronic endoscope according to an embodiment of the present disclosure;

FIG. 7A is a schematic structural diagram I of a connector of an electronic endoscope according to an embodiment of the present disclosure;

FIG. 7B is a schematic structural diagram II of the connector of the electronic endoscope according to the embodiment of the present disclosure;

FIG. 8A is a three-dimensional structural schematic diagram of a plastic deformation limiter of an electronic endoscope according to an embodiment of the present disclosure before use;

FIG. 8B is a three-dimensional structural schematic diagram of the plastic deformation limiter of the electronic endoscope according to the embodiment of the present disclosure after use;

FIG. 9A is a side schematic structural diagram of the plastic deformation limiter of the electronic endoscope according to the embodiment of the present disclosure before use;

FIG. 9B is a side schematic structural diagram of the plastic deformation limiter of the electronic endoscope according to the embodiment of the present disclosure after use;

FIG. 10A is a cross-sectional schematic structural diagram of the plastic deformation limiter of the electronic endoscope according to the embodiment of the present disclosure before use;

FIG. 10B is a cross-sectional schematic structural diagram of the plastic deformation limiter of the electronic endoscope according to the embodiment of the present disclosure after use;

FIG. 11A is a three-dimensional structural schematic diagram of a plastic deformation limiter of an electronic endoscope according to another embodiment of the present disclosure before use;

FIG. 11B is a three-dimensional structural schematic diagram of the plastic deformation limiter of the electronic endoscope according to another embodiment of the present disclosure after use;

FIG. 12A is a side schematic structural diagram of the plastic deformation limiter of the electronic endoscope according to another embodiment of the present disclosure before use;

FIG. 12B is a side schematic structural diagram of the plastic deformation limiter of the electronic endoscope according to another embodiment of the present disclosure after use;

FIG. 13 is a schematic diagram of a spring leaf means in a released state after a change in state of the plastic deformation limiter of the electronic endoscope according to another embodiment of the present disclosure;

FIG. 14 is an axially cross-sectional schematic structural diagram of a connector of an electronic endoscope according to an embodiment of the present disclosure;

FIG. 15 is a radially cross-sectional schematic structural diagram of the connector of the electronic endoscope according to the embodiment of the present disclosure;

FIG. 16 is a schematic structural diagram of a locking rack of an electronic endoscope according to an embodiment of the present disclosure;

FIG. 17 is a schematic structural diagram of a locking driving component of an electronic endoscope according to an embodiment of the present disclosure; and

FIG. 18 is a schematic structural diagram of a high-strength sealed connection of an electronic endoscope according to an embodiment of the present disclosure.

### LIST OF REFERENCE NUMERALS

1, endoscope body; 2, connector; 3, handle; 4, cable; 5, display; 11, inner endoscope rod; 12, outer endoscope tube; 13, limiter; 111, camera module; 112, illumination module; 113, mounting bracket; 114, hollow rod; 115, connecting cable; 122, protective glass; 1131, illumination module mounting hole; 1132, electrical insulation structure; 1133, flat heat conduction surface; 1134, camera module mounting hole; 1141, flat heat conduction surface; 1161, thread structure; 1162, thread structure; 1163, plug-in port; 131a, spring leaf; 132a, elastic piece limiting structure; 25a, assembled object; 251a, assembling acting surface; 131b, hook; 132b, elastic piece limiting structure; 133, elastic piece; 25b, assembled object; 251b, acting surface; 21, locking rack; 22, locking driving component; 23, locking component; 24, ball; 211, limiting slot; 212, ball slot; 213, limiting groove; 214, fitting surface; 221, force arm structure; 222, sliding surface; 223, limiting step; 224, locking surface; 225, transitional surface; 226, unlocking surface; 31, end cap; and 32, housing.

### DETAILED DESCRIPTION OF THE EMBODIMENTS

In order to make the objective, technical solutions, and advantages of the present disclosure clearer, the present disclosure is further described in detail below with reference to the accompanying drawings and embodiments. It should be understood that the specific embodiments described herein are intended merely to explain the present disclosure, rather than to limit the present disclosure.

It should be noted that the drawings provided in the embodiments of the present disclosure merely illustrate the basic concepts of the present disclosure schematically. Therefore, the drawings only show components related to the present disclosure rather than being drawn according to the number, shapes, and sizes of components in actual implementation. The patterns, number, and proportions of components in actual implementation may be changed randomly, and the component layout may be more complex.

In the present disclosure, it should also be noted that the terms such as “center”, “top”, “bottom”, “left”, “right”, “vertical”, “horizontal”, “inner” and “outer”, if used, indicate the orientation or position relationships based on the drawings. These terms are merely intended to facilitate description of the present disclosure and simplify the description, rather than to indicate or imply that the mentioned device or element must have a specific orientation and must be constructed and operated in a specific orientation. Therefore, these terms should not be construed as a limitation to the present disclosure. Moreover, terms such as “first” and “second”, if used, are merely intended for the purpose of description, and should not be construed as indicating or implying relative importance.

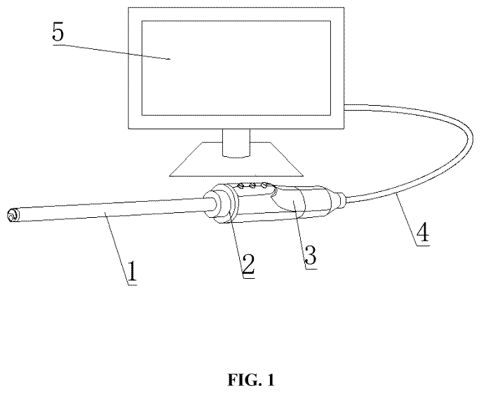

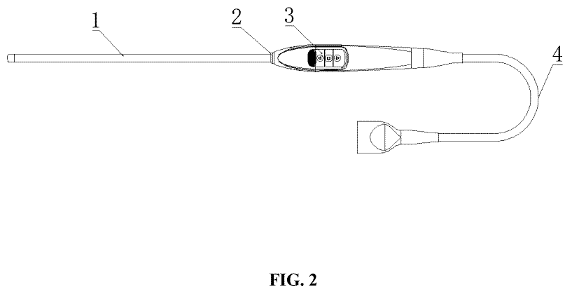

As shown in FIGS. 1 and 2, an embodiment of the present disclosure provides a highly integrated detachable electronic endoscope including an endoscope body 1 and a handle 3 that are detachably connected via a connector 2. The endoscope body 1 is configured for insertion into an examined body, and a camera module 111 and an illumination module 112 are disposed within a front end of the endoscope body 1. An image processing module is disposed inside the handle 3, while an operation key is disposed outside the same, and the handle 3 is connected to an external display 5 via a cable 4. A limiter or an electronic control component allowing customization of a number of uses is disposed on the endoscope body 1, the handle 3, or the connector. The highly integrated detachable electronic endoscope of this embodiment does not need to be connected to an external cold light source and an image processing host, and only needs to be connected to a display screen. The cable 4 includes therein a power cord and a signal line.

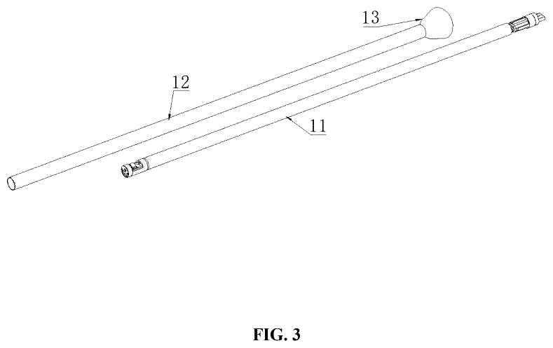

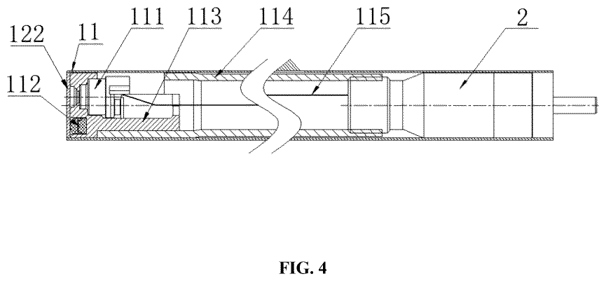

Further, as shown in FIGS. 3 and 4, in this embodiment, the endoscope body 1 includes an outer endoscope tube 12 and an inner endoscope rod 11 independent of each other and capable of being assembled together. The outer endoscope tube 12 has a front end on which protective glass 122 is hermetically mounted, and an open rear end. The inner endoscope rod 11 includes a front end serving as a mounting bracket 113, a hollow rod 114 in a middle portion, and a rear end serving as the connector 2 for connection with the handle 3. The camera module 111 and the illumination module 112 are mounted at a front end of the mounting bracket 113 and limited by the protective glass 122. A rear end of the mounting bracket 113 is connected to a front end of the hollow rod 114. The connector 2 has a front end connected to a rear end of the hollow rod 114, a middle portion hermetically mounted at the rear end of the outer endoscope tube 12, and a rear end serving as a plug-in port and located on an outer side of the outer endoscope tube 12. A connecting cable 115 of the camera module 111 and the illumination module 112 passes through the hollow rod 114 and is electrically connected to the connector 2. When the plug-in port is inserted, the camera module 111 and the illumination module 112 are connected to power and thus are enabled for use. The plug-in port is connected to the electronic control component. The number of uses is incremented by one every time when the plug-in port is inserted. After a set number of uses is reached, when the plug-in port is inserted, the electronic control component controls the camera module 111 and the illumination module 112 to be disabled. In this way, the electronic customization of the number of uses is realized.

Further, the front end of the connector 2 is threadedly connected to the rear end of the hollow rod 114; or the hollow rod 114 is made up of two threadedly connected rod sections to enable length adjustment of the inner endoscope rod 11.

As shown in FIG. 4, in this embodiment, a front end of the outer endoscope tube 12 may be a flat and straight surface perpendicular to the axis. The protective glass 122 is hermetically mounted at a front end of the inner endoscope rod 11 by welding (or adhesive bonding). The inner endoscope rod 11 may be a metal tube. The protective glass 122 is preferably sapphire glass with high wear resistance, or may be other optical lens materials. The inner endoscope rod 11 is preferably made of the common 304 stainless steel, or may also be made of other biocompatible materials.

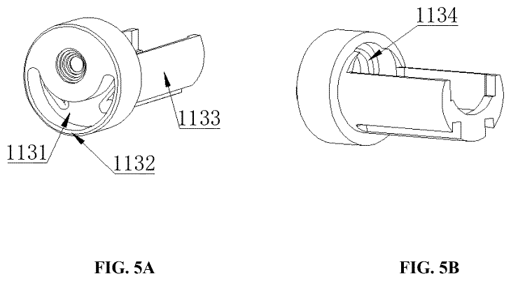

As shown in FIGS. 5A and 5B, a camera module mounting hole 1134 for mounting the camera module 111 and an illumination module mounting hole 1131 for mounting the illumination module 112 are formed in the mounting bracket 113. An outer enclosure of the illumination module mounting hole 1131 is an electrical insulation structure 1132. Preferably, the mounting bracket 113 is an electrically insulating and thermally conductive bracket, and a routing groove is formed in a flat heat conduction surface 1133 thereof.

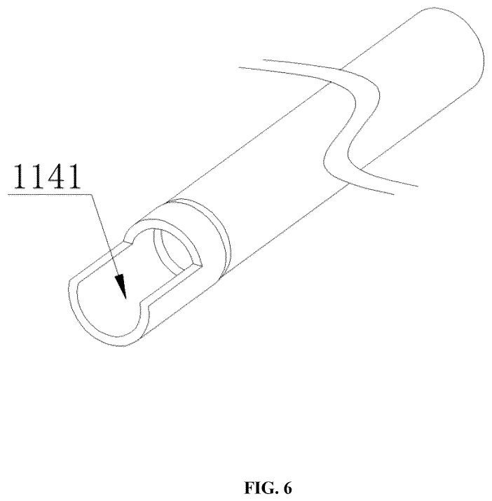

As shown in FIG. 6, the hollow rod 114 is a heat conduction tube. A heat conduction surface 1141 of the hollow rod 114 is in transition fit with the flat heat conduction surface 1133 of the electrically insulating and thermally conductive bracket, thereby not only facilitating assembling, but also not affecting fitting, and guaranteeing the heat conduction effect.

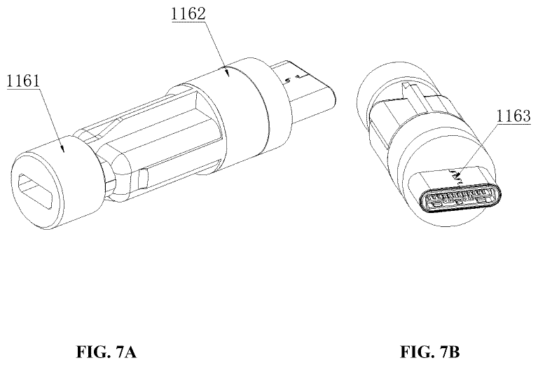

As shown in FIGS. 7A and 7B, the connector 2 is provided with a thread structure 1161 for connection with the hollow rod 114, a thread structure 1162 for adhesive bonding to the outer endoscope tube 12, and a plug-in port 1163 for electrical connection with external power. In this embodiment, the camera module 111 may use an all-plastic lens set, the illumination module 112 may use a light emitting diode (LED) as a light source, the hollow rod 114 may use general-purpose tubing, and the plug-in port 1163 may use a general-purpose type-C interface, thereby reducing the costs. Of course, other materials or types may also be used.

As can be seen, a detachable design is adopted for the endoscope body 1 of this embodiment. All modules can be independent. The inner endoscope rod 11 is adjustable in length and simple to assemble, exhibits high expansibility and portability, and can be applied to different specifications and types of endoscopes. The endoscope body 1 can be customized with a set number of uses. After the set number of uses is reached, the inner endoscope rod 11 can be taken out, and the camera module 111 and the illumination module 112 on the mounting bracket 113 can be recycled. Furthermore, whether the mounting bracket 113, the hollow rod 114, and the connector 2 are recycled can be selected according to an actual situation (if the connector 2 is recycled, its data needs to be reset), thereby further reducing the cost.

The electronic control component in the electronic endoscope of the present disclosure may take a plurality of forms to realize its functionality. In this embodiment, the electronic endoscope may be implemented in one or a combination of the following ways.

In an embodiment of the present disclosure, a circuit is disposed within each of the endoscope body 1 and the handle 3. The electronic control component includes a non-volatile memory chip (e.g., FLASH, an electrically erasable programmable read-only memory (EEPROM), or a ferroelectric random access memory) disposed on the circuit within the endoscope body 1 and a processing chip disposed on the circuit within the handle 3. The non-volatile memory chip is configured to store and encrypt information of a product manufacturer, a product serial number, and a number of uses. The processing chip is configured to: after being installed in the handle 3 and powered on, read and decrypt the information stored on the non-volatile memory chip; if the information meets requirements, activate the camera module 111, and update the information of the number of uses (decrementing a number of allowable uses by one, or incrementing the number of uses by one), and then encrypt and write back the information of the number of uses to the non-volatile memory chip; and if encryption is failed, the information does not meet the requirements, or a number of allowable uses is zero, not activate the camera module 111, or power off the circuit within the endoscope body 1. This way features the highest information security.

For another example, the electronic control component includes a microcontroller unit (MCU) or single-chip microcomputer disposed on the circuit within the endoscope body 1 and a processing chip disposed on the circuit within the handle 3. The MCU or single-chip microcomputer is configured to communicate with the processing chip via a customized communication protocol and to store information of a product manufacturer, a product serial number, and the number of uses. The processing chip is configured to, after being installed in the endoscope body 1 and powered on, read the information stored on the MCU or single-chip microcomputer; if the information meets requirements, activate the camera module 111, and modify the information of the number of uses via a protocol (decrementing a number of allowable uses by one, or incrementing the number of uses by one); and if the information does not meet the requirements or a number of allowable uses is zero, not activate the camera module 111, or power off the endoscope body 1. In this way, since the communication protocol is customized and thus is confidential, the relevant information can be encrypted or not.

For another example, the electronic control component includes a programmable logic device (e.g., a field programmable gate array (FPGA), a complex programmable logic device (CPLD), or an application specific integrated circuit (ASIC)) disposed on the circuit within the endoscope body 1. The programmable logic device includes a circuit of a state machine having preset transition states. The state machine is configured to undergo a transition in state every time when powered on. The transition states are unidirectional. The state machine is further configured to permanently disconnect the circuit within the endoscope body when a transition to a last state occurs so that the endoscope body 1 cannot be used anymore. This way is simplest.

For another example, the electronic control component includes a memory or a microcontroller unit (MCU) disposed on the circuit within the endoscope body 1, and a processing chip and a wireless communication module disposed on the circuit within the handle 3. The memory or the MCU or single-chip microcomputer is configured to store information of a product serial number thereon. The processing chip is configured to, after being installed in the endoscope body 1 and powered on, read the information of the product serial number stored on the memory or the MCU or single-chip microcomputer, and upload the information of the product serial number to a cloud server via the wireless communication module. The cloud server is configured to register the information of the product serial number, and determine whether to distribute a product license according to previous usage logs of the endoscope body 1. If the wireless communication module receives a license to the present electronic endoscope, the wireless communication module transmits the license to the processing chip such that the processing chip activates the camera module 111. If the wireless communication module receives a disable command, the wireless communication module transmits the disable command to the processing chip such that the processing chip does not activate the camera module 111, or powers off the endoscope body 1. In this way, only little information such as the serial number needs to be retained, and the processing chip also does not need to modify and transmit information back.

As shown in FIG. 3, if the number of uses is customized using a mechanical structure, the above-mentioned electronic customization way in the above embodiment is replaced with the customization way by providing the limiter 13 allowing customization of a number of uses. In one embodiment of the present disclosure, the limiter 13 allowing customization of a number of uses may be arranged on the connector 2, and includes a customization tool of a mechanical structure and an acting member. The customization tool includes a movable component capable of naturally expanding and a limiting component capable of retracting and limiting the movable component. When naturally expanding, the movable component is capable of hindering assembling of the connector 2. When the movable component is retracted and limited by the limiting component, the connector can be assembled smoothly. Before assembling each time, the movable component is retracted and limited by the limiting component. During assembling, the acting member acts upon the limiting component such that the limiting component releases the movable component.

The limiting component may be disposable and replaceable; and during assembling, the limiting component is directly damaged by the acting member and thus is released.

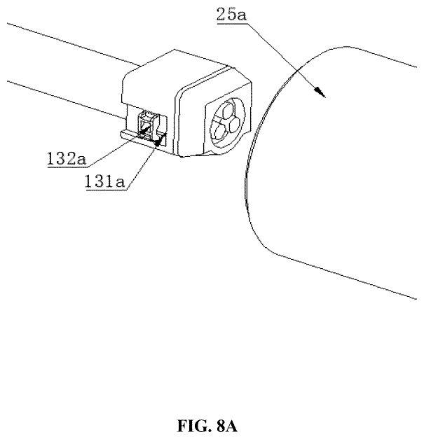

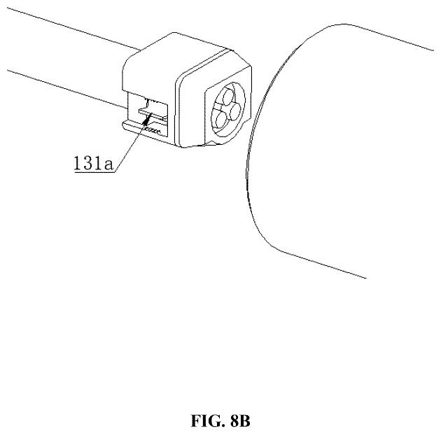

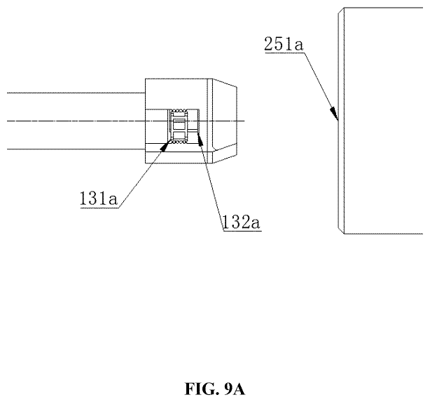

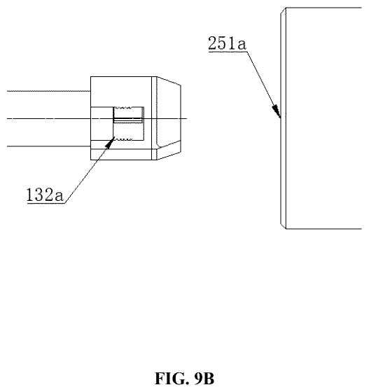

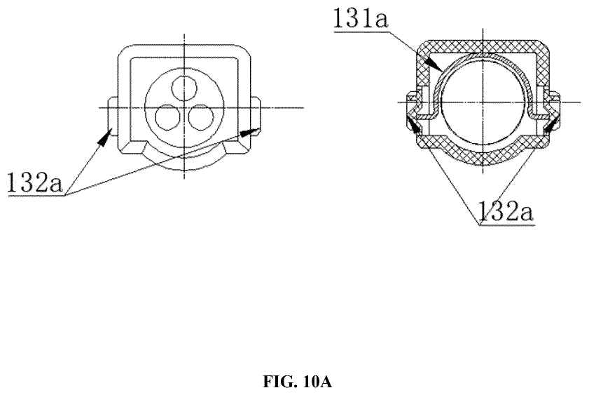

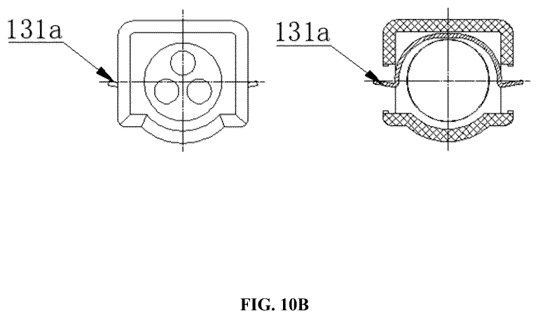

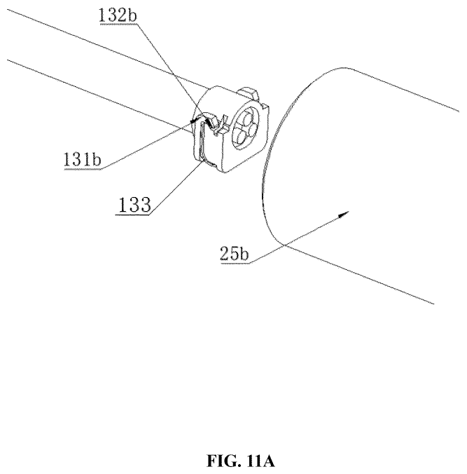

As shown in FIGS. 8A, 8B, 9A, 9B, 10A, and 10B, in this embodiment, the limiting component is an elastic piece limiting structure 132a, the movable component is a spring leaf 131a, and the acting member may be an assembling acting surface 251a of an assembled object 25a for the connector 2. Before assembling, the spring leaf 131a is located within and blocked by the elastic piece limiting structure 132a. During assembling in place, the assembling acting surface 251a props against and removes the elastic piece limiting structure 132a. At this point, the spring leaf 131a is compressed by the inner wall of the assembling acting surface 251a. When being taken out after assembling, the spring leaf 131a bounces off, preventing immediate assembling. In order to enable assembling again, the elastic piece limiting structure 132a must be replaced with a new one. Before assembling, both ends of the spring leaf 131a are separately blocked by the elastic piece limiting structure 132a, avoiding the application of a force on a single side during assembling. An extension direction of both sides of the elastic piece limiting structure 132a is the same as an assembling direction of the connector 2, thereby facilitating the removal of the elastic piece limiting structure 132a during assembling.

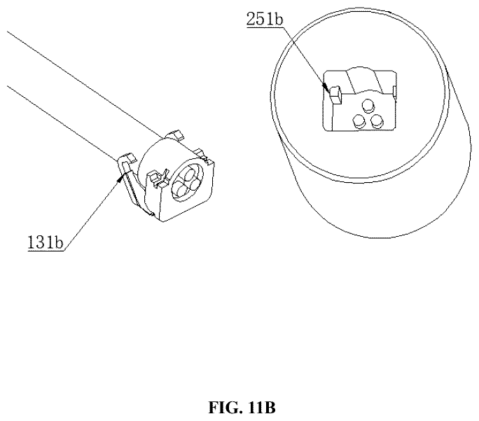

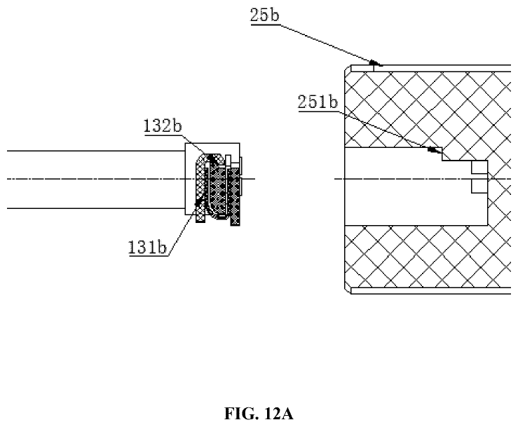

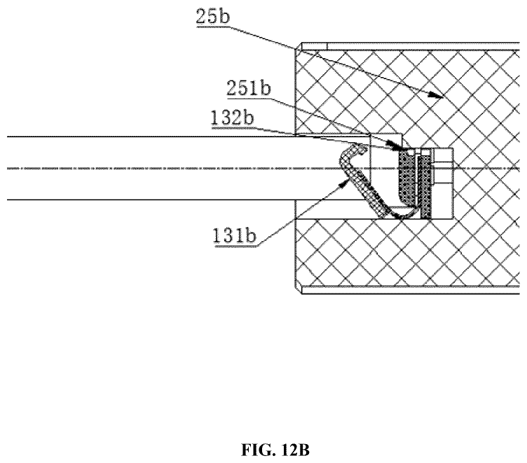

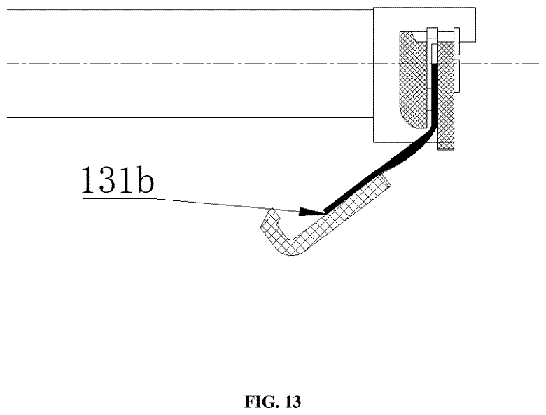

In another embodiment of the present disclosure, as shown in FIGS. 11A, 11B, 12A, 12B, and 13, in the present embodiment, the limiting component is an elastic piece limiting structure 132b in the shape of a slot. The movable component is composed of a spring leaf 133 and a hook 131b connected to the spring leaf 133, and the acting member is an acting surface 251b within an assembling hole of an assembled object 25b for the connector 2. Before assembling, the hook 131b is hooked in the elastic piece limiting structure 132b such that the spring leaf 133 is retracted and limited. During assembling, the hook 131b is pushed out of the elastic piece limiting structure 132b by the acting surface 251b. At this point, the hook 131b is limited by the inner wall of the assembling hole of the assembled object 25b. When being taken out after assembling, the spring leaf 133 pushes away the hook 131b via an elastic force, preventing immediate assembling. In order to enable assembling again, the state of the limiting component must be adjusted using a special tool or manually such that the limiting component is reset. That is, the hook 131b is hooked in the elastic piece limiting structure 132b. The best is to enable resetting using the special tool, thereby avoiding operation without permission. For example, the members are designed to be tiny or be hidden in a shell mounted using non-standard screws. Thus, the special tool must be used. The movable component is located on two sides of an end of the connector 2, and matches the limiting component, avoiding the application of a force on a single side during assembling.

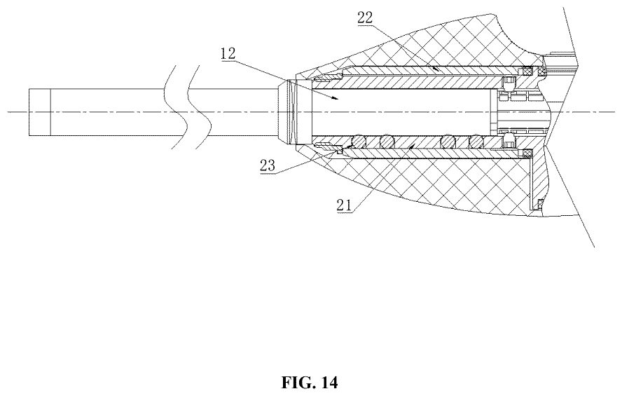

As shown in FIG. 14, in an embodiment of the present disclosure, the connector 2 includes a locking mechanism for locking the endoscope body 1.

In this embodiment, the locked part of the endoscope body 1 is cylindrical (i.e., the outer endoscope tube 12). The locking mechanism includes a locking rack 21, a locking driving component 22, and a locking component 23.

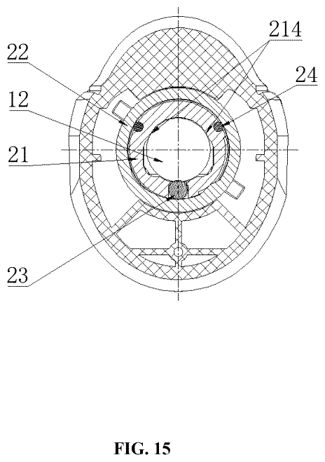

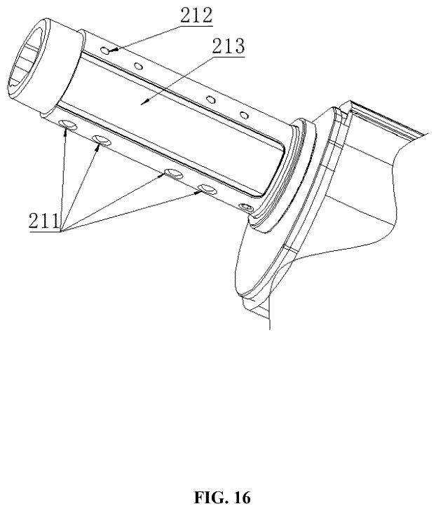

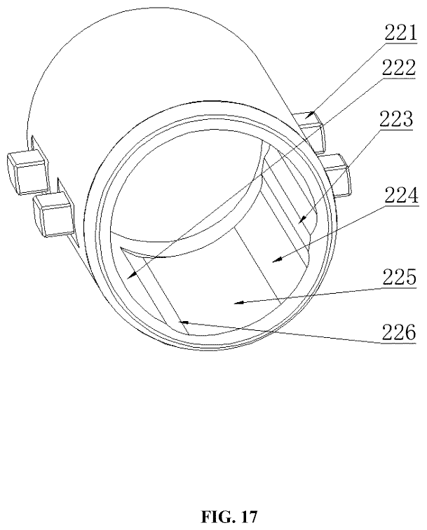

As shown in FIGS. 15 to 17, the locking rack 21 is provided with a through-hole cavity which has a cross-sectional size matching an external size of the outer endoscope tube 12. In a cross-sectional direction, a plurality of fitting surfaces 214 are present between the outer endoscope tube and the through-hole cavity of the locking rack 21.

The locking driving component 22 is sleeved outside the locking rack 21 in a rotary connection manner. An inner wall of the locking driving component 22 is provided with a locking surface 224 and an unlocking surface 226 in a rotation direction. A minimum spacing between the locking surface 224 and the outer endoscope tube 12 is smaller than a minimum spacing between the unlocking surface 226 and the outer endoscope tube 12.

Regarding the locking component 23, a limiting slot 211 radially penetrating through the locking rack 21 is formed in the locking rack 21, and the locking component 23 is placed in the limiting slot 211 and is capable of moving radially within the limiting slot 211. A size of the locking component 23 is between the minimum spacing between the locking surface 224 and the outer endoscope tube 12, and the minimum spacing between the unlocking surface 226 and the outer endoscope tube 12. In a process of the locking driving component 22 rotating on the locking rack 21, a surface of the locking driving component 22 corresponding to the limiting slot 211 is switched between the locking surface 224 and the unlocking surface 226, thereby achieving locking and unlocking of the outer endoscope tube 12.

In this embodiment, the locking component 23 is disposed on the locking rack 21. By rotating the locking driving component 22, the surface of the locking driving component 22 corresponding to the limiting slot 211 can be switched between the locking surface 224 and the unlocking surface 226. Since the minimum spacing between the locking surface 224 and the outer endoscope tube 12 is smaller than the minimum spacing between the unlocking surface 226 and the outer endoscope tube 12, when the surface of the locking driving component 22 corresponding to the limiting slot 211 is switched from the unlocking surface 226 to the locking surface 224, the locking component 23 is gradually clamped between the locking surface 224 and the outer endoscope tube 12. At this point, the locking component 23 exerts a certain pressure on the outer surface of the outer endoscope tube 12, and the locking component 23 works in cooperation with the locking rack 21, together with the plurality of fitting surfaces of the outer endoscope tube 12, to lock the endoscope body 1. Conversely, when the surface of the locking driving component 22 corresponding to the limiting slot 211 is switched from the locking surface 224 to the unlocking surface 226, the locking component 23 cannot come into contact with the unlocking surface 226 and the outer endoscope tube 12 at the same time, and exerts no pressure on the outer endoscope tube 12, and the outer endoscope tube 12 can move within the locking rack 21. Based on the aforementioned measured, the endoscope body 1 can be unlocked and locked rapidly. Moreover, the locking force is provided by the structural component, and is large, allowing for stable locking performance. In addition, this endoscope has the advantages of simple structure, integrated function of components, a reduced total number of components, and high reliability.

Further, at least two groups of locking components 23 and limiting slots 211 matching the locking components 23 are arranged in a length direction of the outer endoscope tube 12. Since the locking component 23 is equivalently in “point contact” with the outer endoscope tube 12, if only a single “point contact” is present, the locked component may swing within the locking rack 21 concentrically with the “point contact”. If at least two groups of locking components 23 and limiting slots 211 are provided, it is equivalent that at least two “point contacts” are provided in the length direction of the outer endoscope tube 12. With the cooperation of a plurality of “point contacts”, the locked component can be avoided from swinging within the locking rack 21, thereby improving the stability of the whole endoscope in the locked state.

Preferably, the locking component 23 is a locking steel ball, and the limiting slot 211 is cylindrical (or approximately cylindrical). Each group of locking components 23 includes at least one locking steel pillar. In the illustrated embodiment, each group of locking components 23 includes two locking steel balls. The locking component 23 is spherical, thereby reducing the friction of motion. In the rotation process of the locking driving component 22, the surface of the locking driving component 22 corresponding to the limiting slot 211 is gradually switched from the unlocking surface 226 to the locking surface 224, and the spacing between the inner wall of the locking driving component 22 and the outer endoscope tube 12 decreases gradually until it is equal to the diameter of the locking steel ball. The locking driving component 22 is rotated continuously. At this point, the locking steel ball is clamped between the inner wall of the locking driving component 22 and the outer endoscope tube 12 and exerts a certain pressure on the outer endoscope tube 12, thereby achieving locking of the outer endoscope tube 12 (with the frictional force between the locking steel ball and the outer endoscope tube 12, natural loosening of the locking steel ball can be effectively prevented).

Preferably, the locking component 23 is made of a material with high compression resistance and hardness such as steel and ceramic.

Further, a transitional surface 225 is provided between the locking surface 224 and the unlocking surface 226. The surface of the locking driving component 22 corresponding to the limiting slot 211 is switched among the unlocking surface 226, the transitional surface 225, and the locking surface 224. If there is no transitional surface 225, in the movement process of the locking driving component 22, the locking steel ball may be stuck at the joint of the locking surface 224 and the unlocking surface 226. If there is the transitional surface 225, it is guaranteed that the locking steel ball is capable of sliding smoothly on the locking surface 224, the transitional surface 225, and the unlocking surface 226, thereby improving the smoothness of the device in use.

In some embodiments as described above, the transitional surface 225 is a smooth transitional surface in the form of a monotonous curve tangent to a circular arc. Cross sections at the joint of the transitional surface 225 and the unlocking surface 226 and the joint of the transitional surface 225 and the locking surface 224 are circular-arc-shaped.

In some other embodiments as described above, when a low requirement is imposed on the transitional surface 225, the transitional surface 225 is in the form of other transition curves such as linear transition. However, in this case, motion sticking (due to the presence of the circular arc) may occur in the movement process. Such a transitional surface may provide a poor hand feeling, and may be worn (without a circular arc).

Further, a plurality of ball slots 212 are formed in an outer wall of the locking rack 21. Balls 24 matching the ball slots 212 are disposed within the ball slots 212. The locking driving component 22 is provided with a sliding surface 222 for the balls 24 to roll thereon. With the balls 24, the frictional force between the locking driving component 22 and the locking rack 21 can be reduced.

Further, at least two fitting surfaces 214 are present between the outer endoscope tube 12 and the through-hole cavity of the locking rack 21. In the illustrated embodiment, two fitting surfaces 214 are adopted, and the two fitting surfaces 214 and the locking component 23 are located in three directions around the outer endoscope tube 12, respectively. When the surface of the limiting slot 211 corresponding to the locking driving component 22 rotates to the locking surface 224, the outer endoscope tube 12 is jointly by the two fitting surfaces 214 and the locking steel ball, thereby achieving clamping of the endoscope body 1.

Further, the highly integrated detachable electronic endoscope further includes a limiting groove 213 and a limiting step 223 matching each other. The limiting groove 213 is located in the outer wall of the locking rack 21, while the limiting step 223 is fixedly arranged on the inner wall of the locking driving component 22, and the limiting step 223 and the limiting groove 213 are arranged axially. The limiting groove 213 is located on the right side of the locking component 23, while the limiting step 223 is located on the right side of the bottom of the locking surface 224 (or the limiting groove 213 is located on the left side of the locking component 23, while the limiting step 223 is located on the left side of the bottom of the locking surface 224, depending on the operation and rotation direction of the locking driving component 22). With the limiting step 223 and the limiting groove 213, the rotation range of the locking driving component 22 on the locking rack 21 is restricted, avoiding that the locking driving component 22 rotates in a full circle on the locking rack 21 and that the locking driving component 22 is self-unlocked after rotating by one circle on the locking rack 21

Two groups of force arm structures 221 are disposed on the locking driving component 22 and are distributed on left and right sides of an outer wall of the locking driving component 22. The locking driving component 22 is capable of increasing arm of force of the force arm structures 221 such that an external force required by a locking operation is reduced.

According to the locking and unlocking operations on the endoscope body 1, the locking driving component 22 located on the locking rack 21 is rotated such that the surface of the locking driving component 22 corresponding to the limiting slot 211 is switched between the unlocking surface 226 and the locking surface 224. Since the minimum spacing between the locking surface 224 and the outer endoscope tube 12 is smaller than the minimum spacing between the unlocking surface 226 and the outer endoscope tube 12, when the surface of the locking driving component 22 corresponding to the limiting slot 211 is switched from the unlocking surface 226 to the locking surface 224, the locking component 23 is gradually clamped between the locking surface 224 and the outer endoscope tube 12. At this point, the locking component 23 exerts a certain pressure on the outer surface of the outer endoscope tube 12, and the locking component 23 works in cooperation with the locking rack 21, together with the plurality of fitting surfaces 214 of the outer endoscope tube 12, to lock the endoscope body 1. Conversely, when the surface of the locking driving component 22 corresponding to the limiting slot 211 is switched from the locking surface 224 to the unlocking surface 226, the locking component 23 cannot come into contact with the unlocking surface 226 and the outer endoscope tube 12 at the same time, and exerts no pressure on the outer endoscope tube 12, and the outer endoscope tube 12 can move within the locking rack 21. Based on the aforementioned measured, the endoscope body 1 can be unlocked and locked rapidly. Moreover, the locking force is provided by the structural component, and is large, allowing for stable locking performance. In addition, this endoscope has the advantages of simple structure, integrated function of components, a reduced total number of components, and high reliability.

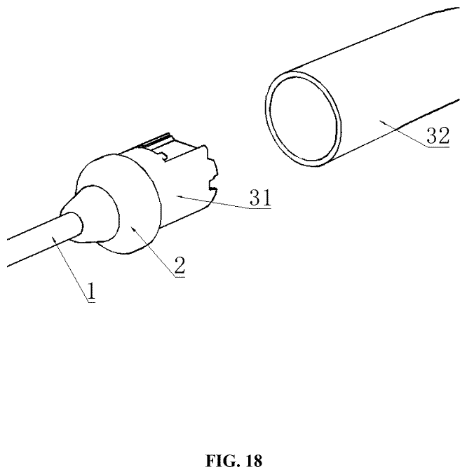

As shown in FIG. 18, the handle 3 is divided into two parts: an end cap 31 and a housing 32. The housing 32 and the end cap 31 are provided with connecting conical surfaces fitting with each other, thereby enabling self-centering mounting and reducing the risk of mismatching. Moreover, the housing 32 and the end cap 31 are provided with mounting holes for preloaded screws. With a tensioning force of the screws and the conical surfaces, a wedge structure can be realized, thereby increasing the degree of stability of fitting. Finally, a glue is applied between the connecting conical surfaces for sealing and material bonding.

It should be pointed out that, based on needs of implementation, each step/component described in the present disclosure can be divided into more steps/components, or two or more steps/components or some operations of the steps/components can be combined into a new step/component to achieve the objective of the present disclosure.

The serial number of each step in the above embodiments does not indicate the order of performing the process. The order of performing each process is determined by its functionality and internal logic, and should not limit the implementation of the embodiments of the present disclosure.

It should be understood that those of ordinary skill in the art can make improvements or transformations based on the above description, and all these improvements and transformations should fall within the protection scope of the appended claims of the present disclosure.

What is claimed is:

1. An integrated detachable electronic endoscope, comprising: an endoscope body and a handle that are detachably connected via a connector;
wherein the endoscope body is configured for insertion into an examined body, and a camera module and an illumination module are disposed within a front end of the endoscope body;
an image processing module is disposed inside the handle, while an operation key is disposed outside the handle, and the handle is connected to an external display via a cable; and
a limiter or an electronic control component allowing customization of a number of uses is disposed on the endoscope body, the handle, or the connector.

2. The integrated detachable electronic endoscope according to claim 1, wherein the endoscope body comprises an outer endoscope tube and an inner endoscope rod independent of each other and capable of being assembled together; the outer endoscope tube has a front end on which protective glass is hermetically mounted, and an open rear end; the inner endoscope rod comprises a front end serving as a mounting bracket, a hollow rod in a middle portion, and a rear end serving as the connector for connection with the handle; the camera module and the illumination module are mounted at a front end of the mounting bracket and limited by the protective glass; a rear end of the mounting bracket is connected to a front end of the hollow rod; the connector has a front end connected to a rear end of the hollow rod, a middle portion hermetically mounted at the rear end of the outer endoscope tube, and a rear end serving as a plug-in port and located on an outer side of the outer endoscope tube; a cable of the camera module and the illumination module passes through the hollow rod and is electrically connected to the connector; when the plug-in port is inserted, the camera module and the illumination module are connected to power and thus are enabled for use; the plug-in port is connected to the electronic control component; a number of uses is incremented by one every time when the plug-in port is inserted; and after a set number of uses is reached, when the plug-in port is inserted, the electronic control component controls the camera module and the illumination module to be disabled.

3. The integrated detachable electronic endoscope according to claim 2, wherein the front end of the connector is threadedly connected to the rear end of the hollow rod; or the hollow rod is made up of two threadedly connected rod sections to enable length adjustment of the inner endoscope rod.

4. The integrated detachable electronic endoscope according to claim 2, wherein the mounting bracket is an electrically insulating and thermally conductive bracket, and the hollow rod is a heat conduction tube.

5. The integrated detachable electronic endoscope according to claim 1, wherein a circuit is disposed within each of the endoscope body and the handle; the electronic control component comprises a non-volatile memory chip disposed on the circuit within the endoscope body and a processing chip disposed on the circuit within the handle; the non-volatile memory chip is configured to store and encrypt information of a product manufacturer, a product serial number, and a number of uses; and the processing chip is configured to: after being installed in the handle and powered on, read and decrypt the information stored on the non-volatile memory chip; when the information meets requirements, activate the camera module, and update the information of the number of uses, and then encrypt and write back the information of the number of uses to the non-volatile memory chip; and when encryption is failed, the information does not meet the requirements, or a number of allowable uses is zero, not activate the camera module, or power off the circuit within the endoscope body.

6. The integrated detachable electronic endoscope according to claim 1, wherein a circuit is disposed within each of the endoscope body and the handle; the electronic control component comprises a microcontroller unit (MCU) or single-chip microcomputer disposed on the circuit within the endoscope body and a processing chip disposed on the circuit within the handle; the MCU or single-chip microcomputer is configured to communicate with the processing chip via a customized communication protocol and to store information of a product manufacturer, a product serial number, and a number of uses; and the processing chip is configured to, after being installed in the endoscope body and powered on, read the information stored on the MCU or single-chip microcomputer; when the information meets requirements, activate the camera module, and modify the information of the number of uses via a protocol; and when the information does not meet the requirements or a number of allowable uses is zero, not activate the camera module, or power off the endoscope body.

7. The integrated detachable electronic endoscope according to claim 1, wherein a circuit is disposed within the endoscope body; the electronic control component comprises a programmable logic device disposed on the circuit within the endoscope body; the programmable logic device comprises a circuit of a state machine having preset transition states; the state machine is configured to undergo a transition in state every time when powered on; the transition states are unidirectional; and the state machine is further configured to permanently disconnect the circuit within the endoscope body when a transition to a last state occurs so that the endoscope body is incapable of being used anymore.

8. The integrated detachable electronic endoscope according to claim 1, wherein a circuit is disposed within each of the endoscope body and the handle; the electronic control component comprises a memory or a microcontroller unit (MCU) or single-chip microcomputer disposed on the circuit within the endoscope body, and a processing chip and a wireless communication module disposed on the circuit within the handle; the memory or the MCU or single-chip microcomputer is configured to store information of a product serial number; the processing chip is configured to, after being installed in the endoscope body and powered on, read the information of the product serial number stored on the memory or the MCU or single-chip microcomputer, and upload the information of the product serial number to a cloud server via the wireless communication module; the cloud server is configured to register the information of the product serial number, and determine whether to distribute a product license according to previous usage logs of the endoscope body;
when the wireless communication module receives a license to the present electronic endoscope, the wireless communication module transmits the license to the processing chip such that the processing chip activates the camera module; and when the wireless communication module receives a disable command, the wireless communication module transmits the disable command to the processing chip such that the processing chip does not activate the camera module, or powers off the endoscope body.

9. The integrated detachable electronic endoscope according to claim 1, wherein the limiter allowing customization of a number of uses is arranged on the connector, and comprises a customization tool of a mechanical structure and an acting member;
the customization tool comprises a movable component capable of naturally expanding and a limiting component capable of retracting and limiting the movable component; when naturally expanding, the movable component is capable of hindering assembling of the connector; when the movable component is retracted and limited by the limiting component, the connector is capable of being assembled smoothly; before assembling each time, the movable component is retracted and limited by the limiting component; and during assembling, the acting member acts upon the limiting component such that the limiting component releases the movable component.

10. The integrated detachable electronic endoscope according to claim 9, wherein the limiting component is disposable and replaceable; and during assembling, the limiting component is directly damaged by the acting member and thus is released.

11. The integrated detachable electronic endoscope according to claim 9, wherein the limiting component has an elastic piece limiting structure, the movable component is a spring leaf, and the acting member is an outer end face of an assembled object assembled with the connector; when the assembled object is not assembled with the connector, the spring leaf is blocked by the elastic piece limiting structure; and during assembling, the outer end face of the assembled object props against and removes the elastic piece limiting structure.

12. The integrated detachable electronic endoscope according to claim 9, wherein the limiting component is reusable and resettable; and during assembling, a state of the limiting component is changed by the acting member, enabling releasing by the limiting component.

13. The integrated detachable electronic endoscope according to claim 9, wherein the limiting component is a hooking slot, the movable component is composed of a spring leaf and a hook connected to the spring leaf, and the acting member is an acting surface of an assembled object assembled with the connector;
when the assembled object is not assembled with the connector, the hook is hooked in the hooking slot such that the spring leaf is retracted and limited; and during assembling, the hook is pushed out of the hooking slot by the acting surface of the assembled object.

14. The integrated detachable electronic endoscope according to claim 1, wherein the connector comprises a locking mechanism for locking the endoscope body, and the locking mechanism comprises:
a locking rack provided with a through-hole cavity, wherein the through-hole cavity has a cross-sectional size matching an external size of the endoscope body; and
in a cross-sectional direction, a plurality of fitting surfaces are present between the endoscope body and the through-hole cavity of the locking rack;
a locking driving component sleeved outside the locking rack in a rotary connection manner, wherein an inner wall of the locking driving component is provided with a locking surface and an unlocking surface in a rotation direction; and a minimum spacing between the locking surface and the endoscope body is smaller than a minimum spacing between the unlocking surface and the endoscope body; and
a locking component, wherein a limiting slot radially penetrating through the locking rack is formed in the locking rack, and the locking component is placed in the limiting slot and is capable of moving radially within the limiting slot; a size of the locking component ranges between the minimum spacing between the locking surface and the endoscope body and the minimum spacing between the unlocking surface and the endoscope body; and in a process of the locking driving component rotating on the locking rack, a surface of the locking driving component corresponding to the limiting slot is switched between the locking surface and the unlocking surface, thereby achieving locking and unlocking of the endoscope body.

15. The integrated detachable electronic endoscope according to claim 14, wherein at least two groups of locking components and limiting slots matching the locking components are arranged in a length direction of the endoscope body.

16. The integrated detachable electronic endoscope according to claim 14, wherein the locking component is a locking steel ball, and the limiting slot is cylindrical.

17. The integrated detachable electronic endoscope according to claim 14, wherein a transitional surface is provided between the locking surface and the unlocking surface; and the surface of the locking driving component corresponding to the limiting slot is switched among the unlocking surface, the transitional surface, and the locking surface.

18. The integrated detachable electronic endoscope according to claim 14, wherein a plurality of ball slots are formed in an outer wall of the locking rack; balls matching the ball slots are disposed within the ball slots; and the locking driving component is provided with a sliding surface for the balls to roll.

19. The integrated detachable electronic endoscope according to claim 14, further comprising a limiting step and a limiting groove matching each other, wherein the limiting groove is located in the outer wall of the locking rack, while the limiting step is fixedly arranged on the inner wall of the locking driving component, and the limiting step and the limiting groove are arranged axially; and the limiting groove and the limiting step are respectively located on a same side of the locking rack and the locking driving component.

20. The integrated detachable electronic endoscope according to claim 14, wherein two groups of force arm structures are disposed on the locking driving component and are distributed on left and right sides of an outer wall of the locking driving component; and the locking driving component is capable of increasing arm of force of the two groups of force arm structures such that an external force required by a locking operation is reduced.
